(19) United States
(12) Patent Application Publication
(10) Pub. No.: US 2015/0209442 A1
(43) Pub. Date: Jul. 30, 2015

(54) METHOD TO IMPROVE PHARMACOKINETICS OF DRUGS

(71) Applicant: The Board of Regents of the University of Texas System, Austin, TX (US)

(72) Inventor: Kenneth M. Hargreaves, San Antonio, TX (US)

(21) Appl. No.: 14/425,105

(22) PCT Filed: Aug. 30, 2013
(86) PCT No.: PCT/US2013/057510

§ 371 Date: Mar. 2, 2015

Related U.S. Application Data
(60) Provisional application No. 61/696,054, filed on Aug. 31, 2012.

(51) Int. Cl. A61K 47/48; A61K 45/06; A61K 39/395; C07K 16/44; C07K 14/46

(52) U.S. Cl. CPC A61K 47/48246; C07K 16/44; C07K 14/46; A61K 39/39533; A61K 45/06; C07K 2319/31

(57) ABSTRACT

A compound comprises a pharmacologically active agent coupled to a plasma protein binding agent. The pharmacologically active agent, in some embodiments, may be an OLAM inhibitor. The plasma protein binding agent, in some embodiments, is a compound that is pharmacologically active in inflamed/injured tissue. A pharmaceutical composition that includes these compounds may be used to treat pain, shock, inflammatory conditions, or combinations thereof in a subject comprising administering to a subject who would benefit from such treatment a therapeutically effective amount of the pharmaceutical composition.

### BACKGROUND OF THE INVENTION

1. Field of the Invention

The present invention generally relates to compositions and methods of improving the pharmacokinetic properties of drugs. More specifically, the present invention relates to compositions and methods of treating inflammation in a subject. 2. Description of the Relevant Art

Inflammation is a protective attempt by the organism to remove the injurious stimuli and to initiate the healing process. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

Acute inflammation is characterized by changes to the vascular system. These changes include vasodilation, increased permeability and the slowing of blood flow. Vasodilation progresses to the capillary level, which brings about a net increase in the amount of blood present. The increased blood causes redness and heat to occur at the site of inflammation. This increased permeability of the vessels results in the movement of plasma into the tissues.

Inflammation can occur during a variety of conditions. Some of the causes of inflammation include burns, chemical irritation, infections, physical injuries (bruising), allergic reactions, radiation (e.g., sunburns), foreign bodies, tumor growth, surgery, and trauma. While inflammation is a common symptom of these conditions, the preferred agent for treatment of each condition is very different. Thus, in order to reduce the symptomatic inflammation, the cause of the inflammation must be addressed. Because inflammation is localized to a specific area of the body, it is desirable to develop methodologies that allow the delivery of the appropriate treatment agent to the area, which may help improve the efficacy of such treatments. In addition, tissues with increased vascular activity may often require specific delivery of drugs. For example, inflammation associated with tumor growth may offer an opportunity for enhanced delivery of therapeutic agents via increased local vascular activity at the tumor site.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic illustration of an inflammatory process; and

FIG. 2 depicts a schematic illustration of a conjugate.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

### Definitions

The terms used throughout this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

As used herein the terms “administration,” “administering,” or the like, when used in the context of providing a pharmaceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered by parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, the term “agonist” generally refers to a type of ligand or drug that binds and alters the activity of a receptor.

As used herein, the term “antagonist” generally refers to a type of receptor ligand which binds a receptor but which does not alter the activity of the receptor; however when used with an agonist, prevents the binding of the agonist to the receptor hence the effect of the agonist.

As used herein, the term “allodynia” generally refers to pain from stimuli which are not normally painful. The pain may occur other than in the area stimulated. Allodynia may generally refer to a heightened pain state.

As used herein, the term “antinociception” generally refers to a reduction in pain sensitivity.

As used herein, the term “monoclonal antibody” generally refers to an antibody obtained from a population of substantially homogeneous antibodies (the individual antibodies comprising the population are identical except for possible naturally occurring mutations that may be present in minor amounts). As used herein, the term “polyclonal antibody” generally refers to a population of antibodies that are directed against a common epitope but which are not identical in structure.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, pharmaceutical compositions, formulations and preparations may include pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

As used herein the term “pharmaceutically acceptable salts” includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

As used herein the term “subject” generally refers to a mammal, and in particular to a human.

As used herein, the term “treat” generally refers to an action taken by a caregiver that involves substantially inhibiting, slowing or reversing the progression of a disease, disorder or condition, substantially ameliorating clinical symptoms of a disease disorder or condition, or substantially preventing the appearance of clinical symptoms of a disease, disorder or condition.

Terms such as “in need of treatment,” “in need thereof,” “benefit from such treatment,” and the like, when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

By “therapeutically effective amount” is meant an amount of a drug or pharmaceutical composition that will elicit at least one desired biological or physiological response of a cell, a tissue, a system, animal or human that is being sought by a researcher, veterinarian, physician or other caregiver.

The term “OLAM inhibitor” is used to describe a compound that inhibits and/or minimizes the production of oxidized linoleic acid metabolites and/or blocks the activity of oxidized linoleic acid metabolites.

### Plasma Protein Coupled Pharmacologically Active Agents

Many drugs have limitations in their pharmacokinetic properties due to rapid renal filtration, degradation by circulating enzymes or poor penetration into inflamed tissue. Among this list of drugs with suboptimal pharmacokinetic properties are aptamers, tioconazole, nifedipine, metoprolol, and others. Many of the major physiological responses of acute inflammation are vascular in nature and include plasma extravasation and vasodilation. Plasma extravasation is the outflow of fluid and plasma proteins into the inflamed extracellular compartment. Therefore, agents that are bound to plasma proteins are likely to exhibit reduced renal filtration, reduced exposure to circulating enzymes and/or increased delivery into inflamed tissues by the process of plasma extravasation.

Many anti-inflammatory drugs are heavily bound to plasma proteins. Indeed, most nonsteroidal anti-inflammatory drugs (NSAIDs), including flurbiprofen are >99% bound to plasma proteins. Thus, only ˜0.1% of the total flurbiprofen concentration in plasma is in the “free” (unbound) state. Because only the free concentration is biologically active, physiological responses that alter delivery of plasma proteins or binding of these drugs to plasma proteins are likely to alter the magnitude of the pharmacological effect.

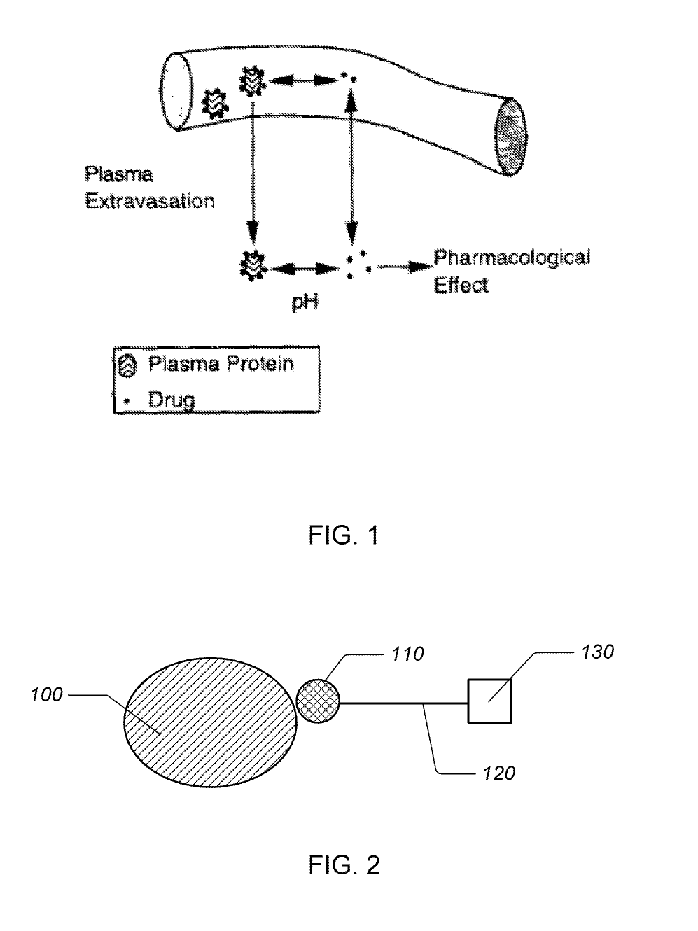

Our studies indicate that: inflammation alters the delivery of drugs to the inflamed tissue; activation of capsaicin-sensitive nerves increases the content of protein-bound drugs; and that reduced pH increases free drug concentrations of the protein-bound drugs. Thus, alterations in both plasma extravasation and tissue pH seem to be relevant factors regulating the delivery and bioavailability of anti-inflammatory drugs and other compounds which are highly protein-bound. FIG. 1 depicts a schematic illustration of an inflammatory process. Drugs that are heavily bound to plasma proteins are released into extracellular space primarily by the process of plasma extravasation. Both vasodilation (e.g., blood flow) and vascular permeability regulate plasma extravasation. The drug is distributed between free (i.e. “unbound”) and bound (i.e. plasma protein:drug complex) states. Only the free drug concentration is pharmacologically active. Factors that influence drug binding to plasma proteins will alter free drug concentration and therefore would be predicted to alter the magnitude of the pharmacological effect. Thus, both plasma extravasation and local tissue ion concentrations such as altered pH in inflamed areas would be expected to alter the efficacy of protein-bound drugs.

Inflammation of tissue and/or activation of capsaicin-sensitive nerves within tissues (tissue pain states) can occur due to a number of different causes, all requiring a specific and different treatment. Many of the treatments involve drugs that may be poorly solubilized, and have limited availability in the sites of inflammation and tissue pain states. A method has been developed that 1) improves pharmacokinetic properties of drugs and 2) enhances delivery of drugs to inflamed, painful or injured tissue. The basic methodology is to conjugate agents with high plasma protein binding properties (a “plasma protein binding compound”) to one or more other selected drug(s) of interest. This will effectively confer increased plasma binding properties to the drug or drugs of interest and result in targeting of agents to body sites where above normal states of vasodilation and vascular permeability (and thus above normal levels of plasma extravasation) exist. A schematic diagram of the conjugate is depicted in FIG. 2. A therapeutic agent of interest 130 may be coupled to a plasma protein 100 through a plasma protein binding compound 110 and, optionally, through a linker 120 which couples the therapeutic agent 130 to the plasma protein binding compound 110.

Examples of diseases or medical conditions involving stressed, inflamed, painful and/or traumatized tissues and cells include, but are not limited to ischemic tissue conditions including ischemic heart disease, myocardial infarction, cancer, burns, traumatic tissue injury, arthritis, surgery-induced tissue damage, infections, cerebrovascular accidents, and other conditions involving a disruption in cellular membrane integrity.

For the treatment of inflammation a number of pharmacologically active agents may be used, depending on the cause of the inflammation. Examples of drug classes used to treat inflammation include, but are not limited to: antibiotics; growth factors; local anesthetics; analgesics; and antihistamines Any pharmacologically active agent from these drug classes may be coupled to a plasma protein binding compound to enhance the delivery of these drugs to the inflamed, painful and/or injured body site. With respect to pain and inflammation management, additional benefits may be gained if the plasma protein binding compound is also an anti-inflammatory compound, or has pain reducing properties. Examples of compounds that are plasma protein binding compounds and are pharmacologically active in inflamed, injured and/or painful tissue include non-steroidal anti-inflammatory drugs (“NSAIDS”), antibiotics, local anesthetics, opiates, opioids, or steroids. Specific examples of NSAIDS that are plasma binding compounds include, but are not limited to: salicylates (e.g., Aspirin (acetylsalicylic acid), diflunisal, and salsalate); propionic acid derivatives (e.g., ibuprofen, dexibuprofen, naproxen, fenoprofen, ketoprofen, dexketoprofen, flurbiprofen, oxaprozin, and loxoprofen); acetic acid derivatives (e.g., indomethacin, tolmetin, sulindac, etodolac, ketorolac, diclofenac, and nabumetone); enolic acid (oxicam) derivatives (e.g., piroxicam, meloxicam, tenoxicam, droxicam, lornoxicam, and isoxicam); and fenamic acid derivatives (fenamates) (e.g., mefenamic acid; meclofenamic acid; flufenamic acid; and tolfenamic acid. Other exemplary plasma protein binding compounds that also exhibit pain reducing properties or anti-inflammatory properties are antibiotics (e.g., clindamycin, erythromycin, or the sulphonamides), local anesthetics (e.g., bupivacaine), opiates (e.g., methadone), or steroids (e.g., prednisolone).

For the treatment of other conditions such as cancer, myocardial infarction, cerebrovascular accidents, ulcers, etc, the increased vascular permeability provides the opportunity for local delivery of high concentrations of drugs that are bound to plasma proteins. Examples of drug classes used to treat these conditions include inhibitors of tissue plasminogen activator, anti-cancer drugs including chemotherapeutics or drugs that alter angiogenesis, anti-ulcer drugs, and so on.

In an embodiment, the plasma protein binding compound, will bind to the plasma proteins in a pH-dependent fashion, such that binding would be reduced at the lower pH values (e.g., at pH of less than 7) seen in tissue inflammation, cancer, injury and tissue hypoxia. This would lead to increased free drug concentrations in the inflamed tissue and therefore improved pharmacodynamics. Examples of drugs that bind to plasma proteins in a pH-dependent fashion are biperiden, clindamycin, dexamethasone, fluoxetine, and nefinavir.

With regard to hypoxia, it is known that hypoxia leads to inflammation in some diseases/disorders involving tissues. Hypoxia in tissues leads to lower pH. As noted above, in some embodiments, the protein binding compound may have reduced binding to the plasma protein at reduced levels. Thus, the use of pH dependent complexes of protein binding compounds and therapeutic agents allows an effective therapy for treating systemic shock and other conditions associated with hypoxic conditions (re-perfusion injuries, decreasing myocardial damage post-myocardial infarction and other conditions).

In another embodiment, plasma bound antioxidants may be used for the treatment of tissue reperfusion injuries caused by free radicals in hypoxic tissues which are suddenly re-oxygenated. For example, antioxidants such as NDGA, Vitamin C, glutathione, resveratrol, vitamin E, β-carotene, and astaxanthin may be bound to a plasma protein either directly or through a plasma protein binding compound to diseases associated with reactive oxygen radical production. Clinical situations where this occurs includes, but is not limited to: 1.) post acute myocardial infarction to save as much stunned and ischemic myocardium as possible; 2.) reperfusion injury post-organ transplant; and 3.) severed limbs which are microsurgically attached.

The pharmacologically active agent used to treat the inflammatory condition may be coupled to the plasma protein binding compounds using a number of techniques. In an embodiment, a pharmacologically active agent may be directly covalently linked to a plasma protein binding compound. For example, protein, peptide, ribonucleotide or nucleotide-based drugs (e.g., antibodies and aptamers) typically include one or more carboxylic acid functional groups and one or more amino functional groups. Any of the reactive carboxylic acid groups or reactive amino groups can be used to form a covalent bond to functional groups on the plasma protein binding compound. For example, many NSAIDS, and other compounds that bind to plasma proteins, have a free carboxylic acid group which can be covalently linked to an aptamer or antibody through an amide linkage. A free amino group of the aptamer or antibody may be linked to the carboxylic acid group using standard reactions for forming amino acid linkages.

Small molecule drugs may also be covalently linked to a plasma protein binding compound. In an embodiment, reactive functional groups on the small molecule drugs may be coupled with reactive functional groups on a plasma protein binding compound. For example, many NSAIDS, and other compounds that bind to plasma proteins, have a free carboxylic acid group which can be covalently linked to reactive alcohol groups, amino groups, or thiol groups on the small molecule drugs.

In some embodiments, it may be desired to use a linker molecule to couple the pharmacologically active agent used to treat the inflammatory condition to a plasma protein binding compound. A linker molecule is generally any molecule that is used to covalently couple the drug to the plasma protein binding compound. In some embodiments, a linker may be a homobifunctional linker. Such compounds may have the general formula R—(CH₂)ₙ—R, where R is CO₂H, NH₂, OH, SH, CH═O, CR¹═O, CH═NH, or halogen; n is 1-10, and R¹ is C₁-C₆ alkyl). Alternatively, the linker may be a heterobifunctional linker. Such compounds may have the general formula R²—(CH₂)ₙ—R³, where R² and R³ are different, and where each R² and R³ is CO₂H, NH₂, OH, SH, CH═O, CR¹═O, CH═NH, or halogen; n is 1-10, and R¹ is C₁-C₆ alkyl. A linker molecule may covalently bond with at least one reactive functional group of the drug and at least one reactive functional group of the plasma protein binding compound. Specific linkers may be chosen for use in the plasma protein—plasma protein binding compound—linker—drug complex such that drug release may be optimized for specific ionic conditions, such as a certain pH or pH range.

In another embodiment, a pharmacologically active agent used to treat the inflammatory condition may be bound to a plasma protein binding compound by a linker molecule that is removed by enzymes present in inflamed tissue. In such embodiments, the linked plasma protein binding compound helps to transport the drug to the inflamed tissue. Once the complex reaches the inflamed tissue, the linker is removed by the enzymes present at the site of inflammation.

In other embodiments, a plasma protein binding compound may be bound to one or more other compounds which may or may not themselves be bound, the resulting structure yielding multi-valent target binding capability. For example, the plasma protein binding compound may be bound to two or more therapeutic compounds, each of the therapeutic compounds being specific for a different target. In this way a single complex may be designed to address multiple biological pathways that contribute to the disease state.

In an embodiment, the plasma protein binding compound may not be a pharmacologically active compound. In such situations, the plasma protein binding compound is simply used to transport the drug into the plasma and to the site of inflammation.

### Enhancement of OLAM Inhibitors

TRPV1, also known as the capsaicin receptor, plays a pivotal role in burn injury and other important pain conditions by evoked hyperalgesia and allodynia such that the mice deficient in TRPV1 protein show little to no heat hyperalgesia in these models. The key role played by TRPV1 in the development of thermal hyperalgesia and possibly mechanical hyperalgesia in various pain models is well established in animal and human studies. Signaling cascades initiated by a variety of inflammatory mediators may sensitize TRPV1 and contribute to inflammatory hyperalgesia. Given the importance of TRPV1 in inflammatory pain, burn pain and cancer pain, including other various pain states, a number of groups in the past have developed antagonists against TRPV 1 as potential treatments for pain and/or inflammatory conditions. Unfortunately, it was discovered that antagonism of the TRPV1 receptor with TRPV1 antagonists can lead to sometimes dangerous levels of hyperthermia. This insight has led to the search for endogenous targets upstream of the TRPV 1 receptor which, if eliminated, immunoneutralized or otherwise interfered with, might alleviate pain and/or inflammatory conditions.

Ours was the first group to demonstrate that oxidized metabolites of linoleic acid act as ligands at the TRP-class of neurons, and in the case of the TRPV1 receptor, Oxidized Linoleic Acid Metabolites (OLAMs) are TRPV1 agonists and mediate pain and/or inflammatory conditions. Linoleic acid is also known by its IUPAC name cis, cis-9,12-octadecadienoic acid. Linoleic acid has a structure:

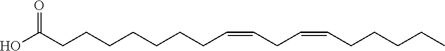 [SMILES: CCCCC/C=C\C/C=C\CCCCCCCC(=O)O]

In some embodiments, pharmacological interventions that can block the generation of the endogenous TRPV1 ligand in response to heat may be of therapeutic use. Oxidized linoleic acid metabolites are generated upon heat stimulation of skin. Oxidized linoleic acid metabolites include, but are not limited to, oxo linoleic acid metabolites, hydroxyl linoleic acid metabolites, and epoxy linoleic acid metabolites. Examples of oxo linoleic acid metabolites include, but are not limited to (10E,12Z)-9-oxooctadeca-10,12-dienoic acid (9-oxoODE, 9-KODE) and (9Z,11E)-13-oxooctadeca-9,11-dienoic acid (13-oxoODE, 13-KODE). Examples of hydroxyl linoleic acid metabolites include, but are not limited to: 9-hydroxyoctadecadienoic acid (9-HODE); 13-hydroxyoctadecadienoic acid (13-HODE); 9(10)-dihydroxy-octadec-12-enoic acid (9,10-DiHOME); and 12,13-dihydroxy-9Z-octadecenoic acid (12,13-DiHOME). Examples of epoxy linoleic acid metabolites include, but are not limited to: (12Z)-9,10-epoxyoctadecenoic acid (9(10)-EpOME) and 12,13-epoxyoctadec-9Z-enoic acid (12(13)-EpOME). It is believed that oxidized linoleic acid metabolites may function as endogenous TRPV1 agonists.

In some embodiments, the blockade of synthesis or immunoneutralization of oxidized linoleic acid metabolites results in decreased activation of pain sensing neurons by heat in vitro and results in thermal antinociception in vivo Immunoneutralization of oxidized linoleic acid metabolites may be accomplished by the use of one or more antibodies that bind to at least one oxidized linoleic acid metabolite. Antibodies for oxidized linoleic acids may be formed using the procedure of Spindler et al. (Spindler et al. “Significance and immunoassay of 9- and 13-hydroxyoctadecadienoic acids.” Biochem Biophys Res Commun. 1996; 218:187-191), which is incorporated herein by reference. Antibodies for oxidized linoleic acids may be monoclonal antibodies or polyclonal antibodies.

In some embodiments, application of a lipoxygenase (LOX) inhibitor (e.g., nordihydroguaiaretic acid (NDGA)) may be effective to treat pain or inflammation. LOX inhibitors may be administered sufficiently to substantially attenuate the catalytic effect of enzymes such as EC 1.13.11.34 (aka: arachidonate 5-lipoxygenase) in order to treat pain, shock, and/or inflammatory conditions.

In some embodiments, a method of treating a pain, shock and/or inflammatory conditions may include administering a cytochrome P-450 (CYP) enzyme inhibitor sufficient to substantially inhibit and/or reduce the catalytic effect of multiple P450 isozymes capable of synthesizing oxidized linoleic acid metabolites (OLAMs). In some embodiments, the CYP inhibitor may be administered intravenously, orally, topically (for burns or wounds), directly into the central nervous system (e.g., epidural), or any other method described herein or that will be known to those skilled in the art. In some embodiments, a method of treating a pain, shock and/or inflammatory conditions may include administering a cytochrome P-450 (CYP) isoenzyme inhibitor sufficient to substantially inhibit or reduce the catalytic effect of enzyme EC 1.14.14.1 (aka: CYP2C9 and CYP2C19).

Examples of CYP inhibitors include, but are not limited to; ketoconazole, miconazole, fluconazole, benzbromarone, sulfaphenazole, valproic acid, amiodarone, cimetidine, fenofibrate, fluvastatin, lovastatin, fluvoxamine, sertraline, isoniazid, probenecid, sulfamethoxazole, teniposide, voriconazole, and zafirlukast. In some embodiments, the CYP inhibitor may be administered intravenously, orally, topically (for burns or wounds), directly into the central nervous system (e.g., epidural), or any other method described herein or that will be known to those skilled in the art.

In one embodiment, cytochrome P450 inhibitors that block the formation of linoleic acid metabolites may be used as analgesic drugs. In one embodiment, ketoconazole is administered topically or systemically to relieve pain or inflammation, shock or hypotension mediated by the formation of linoleic acid metabolites.

In some embodiments, a method of treating a pain, shock, and/or inflammatory condition may include administering an antioxidant sufficient to substantially inhibit and/or reduce the catalytic effect of relevant metabolic enzymes in the Linoleate pathway. In some embodiments, antioxidant inhibitors of relevant metabolic enzymes in the Linoleate pathway may include Nordihydroguaiaretic acid (NDGA), Vitamin E and/or Vitamin E derivatives (e.g., water soluble Vitamin E derivative). NDGA may function at least in part as a therapeutic agent due to its strong antioxidant characteristics.

In some embodiments, the blockade of synthesis or immunoneutralization of oxidized linoleic acid metabolites results in decreased activation of pain sensing neurons by heat in vitro and results in thermal antinociception in vivo Immunoneutralization of oxidized linoleic acid metabolites may be accomplished by the use of one or more aptamers that bind to at least one oxidized linoleic acid metabolite.

Recent research has indicated that activation of TRPV1 by 9-HODE may have other roles in the body depending upon the expression of TRPV1. TRPV1 in the spinal cord may play an important role in maintenance of thermal and mechanical allodynia in inflammatory or other pain conditions. Depolarization of the spinal cord may lead to the release of 9-HODE and activation of TRPV 1. 9-HODE in the spinal cord may lead to development of mechanical allodynia. Similar to heated skin, depolarized spinal cord (with high potassium) may release compound(s) that have TRPV1 agonist activity. Depolarized spinal cord superfusate may contain significantly higher amounts of 9-HODE. Moreover, activation of TRPV 1 in the spinal cord by capsaicin (positive control) or by 9-HODE results in tactile allodynia that is completely reversible by a TRPV1 antagonist. Thus, in some embodiments, the role of 9-HODE and similar linoleic acid oxidation products extends beyond heat-nociception.

In some embodiments, a method may include treating shock and/or inflammation. The therapy used to treat any one case of shock depends upon the cause of the patient's hypoperfusional disorder, however, a disruption in cellular membrane integrity, leading to the release and oxidation of linoleic acid metabolites from stressed cells, is a process common to many if not most cases of shock. These oxidized linoleic acid metabolites have paracrine and/or endocrine effects that act to worsen the symptoms of shock. A method as described herein may effectively delay the multi-organ failure associated with Refractory (Irreversible) shock. This therapeutic method may be used in many, if not most cases of shock and save many lives.

In some embodiments, given the role of these metabolites in various other diseases (e.g., arthritis, pulmonary edema and shock), similar methods and antibodies may be used in treating these conditions.

To improve the pharmacokinetic properties of OLAM inhibitors, an OLAM inhibitor may be coupled to an agent with plasma protein binding properties (“plasma protein binding compounds”). This will confer increased plasma binding properties to the OLAM inhibitor, allowing the OLAM inhibitor to be carried to the site of inflammation through plasma extravasation. Once the plasma protein bound OLAM inhibitor arrives at the site of inflammation, the inhibitor may become unbound from the plasma protein due to the low pH generally associated with tissue inflammation or injury.

In an example ibuprofen, a drug that is more than 99% bound to plasma proteins, may be linked to an aptamer (that acts as an OLAM inhibitor), a class of drugs that demonstrates poor plasma protein binding properties. This combination would decrease renal filtration of the aptamer (since plasma proteins are not filtered), decrease degradation by circulating enzymes, and increase delivery to inflamed tissue due to plasma extravasation of plasma proteins into areas of tissue injury.

In another embodiment, cytochrome P450 inhibitors that block the formation of linoleic acid metabolites (e.g., ketoconazole) may be coupled to a plasma protein through a plasma protein binding compound. This may enhance the effectiveness of the drug and minimize the amount of drug required to achieve the therapeutic effect. For example, it is possible to administer sufficient ketoconazole systemically to alleviate pain if the ketoconazole is bound (via highly protein bound compounds) to serum albumin, and then extravasated to an affected body site where the drug is released and can act as a CYP inhibitor at the affected body site. This is believed to cause an analgesic effect with a significantly smaller dose thus limiting side effects associated with cytochrome P450 inhibitors. Furthermore, the enhanced binding to plasma proteins is believed to improve pharmacokinetics and pharmacodynamics of the bound drug.

### Pharmaceutical Compositions

Any suitable route of administration may be employed for providing a subject with an effective dosage of the compounds described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The compounds described herein may be present in pharmaceutical compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, compositions may be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the compositions with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Soft gelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or soft gelatin capsules, for example, for identification or in order to characterize combinations of active compound doses, or to disguise the capsule contents for usage in clinical or other studies.

In some embodiments, the compounds will typically be formulated in such vehicles at a concentration of about 0.1 mg/ml to 100 mg/ml.

For the prevention or treatment of pain or inflammation, the appropriate dosage of the composition will depend on the type of the severity and location of the pain or inflammation, whether the compositions are administered for preventive or therapeutic purposes, previous therapy, the patient's clinical history and response to the composition, and the discretion of the attending physician. The composition is suitably administered to the patient at one time or over a series of treatments.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A compound comprising:
a pharmacologically active agent;
a plasma protein binding compound coupled to the pharmacologically active agent,
wherein the plasma protein binding compound reversibly binds to plasma proteins when the compound is exposed to plasma proteins in the body of a subject treated with the compound.

2. The compound of claim 1, wherein the pharmacologically active agent is used to treat an inflammatory condition.

3. The compound of claim 1, wherein the pharmacologically active agent is used to treat a pain condition.

4. The compound of claim 1, wherein the pharmacologically active agent is an antibiotic.

5. The compound of claim 1, wherein the pharmacologically active agent is a local anesthetic.

6. The compound of claim 1, wherein the pharmacologically active agent is a growth factor.

7. The compound of claim 1, wherein the pharmacologically active agent is an analgesic.

8. The compound of claim 1, wherein the pharmacologically active agent is an antihistamine.

9. The compound of claim 1, wherein the pharmacologically active agent is an anti-cancer drug.

10. The compound of claim 1, wherein the pharmacologically active agent is an anti-coagulant.

11. The compound of claim 1, wherein the pharmacologically active agent is an anti-ulcer drug.

12. The compound of claim 1, wherein the pharmacologically active agent is an OLAM inhibitor.

13. The compound of claim 1, wherein the pharmacologically active agent is an aptamer that is an OLAM inhibitor.

14. The compound of claim 1, wherein the pharmacologically active agent is an antibody that is an OLAM inhibitor.

15. The compound of claim 1, wherein the pharmacologically active agent is an LOX inhibitor.

16. The compound of claim 1, wherein the pharmacologically active agent is a CYP inhibitor.

17. The compound of claim 1, wherein the pharmacologically active agent is an antioxidant that is an OLAM inhibitor.

18. The compound of any one of claims 1-17, wherein the plasma protein binding compound is pharmacologically active in inflamed/injured tissue.

19. The compound of any one of claims 1-17, wherein the plasma protein binding compound is an NSAID.

20. The compound of any one of claims 1-19, wherein the plasma protein binding compound binds to plasma proteins in a pH-dependent fashion.

21. The compound of any one of claims 1-20, wherein binding of the plasma protein binding compound to plasma proteins is reduced at pH lower than 7.

22. The compound of any one of claims 1-21, further comprising a linker molecule coupling the pharmacologically active agent to the plasma protein binding compound.

23. A pharmaceutical composition comprising one or more compounds as described in any one of claims 1-22.

24. A method of treating pain, shock, inflammatory conditions, or combinations thereof in a subject comprising administering to a subject who would benefit from such treatment a therapeutically effective amount of the pharmaceutical composition of claim 23.

25. A compound comprising a pharmacologically active agent coupled to a plasma protein binding compound.
